(19) United States
(12) Patent Application Publication
(10) Pub. No.: US 2003/0208110 A1
(43) Pub. Date: Nov. 6, 2003

(54) Physiological monitoring using wrist-mounted device

(76) Inventors: James R Mault, Evergreen, CO (US); John Sanderson, Irvine, CA (US)

Correspondence Address: TERRENCE GASPARD, 1343 HOLIDAY PLACE, NEW ORLEANS, LA, 70114

(21) Appl. No.: 10/296,605

(22) PCT Filed: May 25, 2001
(86) PCT No.: PCT/US01/17098

(30) Foreign Application Priority Data
May 25, 2001 PCT/US01/17098

(51) Int. Cl. A61B 5/00

(52) U.S. Cl. 600/300; 128/921; 600/365

(57) ABSTRACT

A wrist-mounted device for assisting a person to maintain a calorie balance goal comprises a processor; a display; a product identifier input mechanism; an activity level input receiving an activity level signal correlated with the physical activity level of the person; a resting metabolic rate input mechanism; a database correlating product identifiers with nutritional data; and a calorie management algorithm, adapted to calculate a calorie balance from the calorie values of foods consumed, the resting metabolic rate, and the activity level signal, and to present the calorie balance to the person on the display. Embodiments of the wirst-mounted device are also useful in blood glucose control.

### FIELD OF THE INVENTION

The invention relates to physiological and health monitoring of a person using a wrist-mounted device.

### BACKGROUND OF THE INVENTION

Conventional weight loss programs fail to properly account for resting energy expenditure of a person. Food consumption by the person using the weight loss program is conventionally recorded in great detail, allowing an accurate calorie intake to be determined. However, weight control is related to the person's net calorie balance, the difference between calorie intake and calorie expenditure. Caloric expenditure is usually not known accurately. It is possible to estimate the calorie expenditure related to various physical activities. However, as discussed by Remmereit in U.S. Pat. No. 6,034,132, for a typical person, 70% of total calorie expenditure is due to their resting metabolic rate (RMR). In a conventional diet program, RMR is estimated from the height, weight, age, and gender of the person, for example using the Harris-Benedict equation. This equation, well known to those skilled in the nutritional arts, is given in U.S. Pat. No. 5,839,901 to Karkanen, and in U.S. Pat. No. 5,639,471 to Chait et al., incorporated herein by reference. There are serious inadequacies in using the Harris-Benedict equation (or any similar equation) in a weight loss program. The Harris-Benedict equation provides only an estimated RMR, which is an average value for people of similar height, weight, age, and gender. However, due to natural variations in physiology, it need not be accurate for a specific individual.

The total calorie expenditure of a person comprises a resting metabolic component and a physical activity component. Total energy expenditure (TEE) is the sum of resting energy expenditure (REE) and activity energy expenditure (AEE), i.e. TEE=AEE+REE. Weight loss occurs if total calorie expenditure (TEE) exceeds total calorie intake over a given time period. The net calorie balance for a person is the difference between calorie expenditure and calorie intake.

Conventional weight loss programs use an estimated TEE based on estimates of activity levels, and estimates of REE from the Harris-Benedict equation. However, if REE is not estimated correctly, the person's calorie balance cannot be known accurately, and the outcome of a weight loss program is likely to be unsatisfactory.

It is also known that RMR often falls during a restricted calorie diet. The Harris-Benedict equation scales RMR with weight, but does not account for a natural slowing of human metabolism in what the body may interpret as partial starvation conditions. Physical activity during the restricted calorie diet may cause RMR to fall further to allow the body to conserve energy, or, alternatively, it may cause RMR to increase due to an increase in muscle mass. Hence, in addition to unpredictable variations in RMR from person to person, there are also unpredictable changes in RMR in response to a weight control program. The improved weight control system described herein overcomes these problems.

Resting metabolic rate (RMR) can be measured using an indirect calorimeter. REE corresponds to the value RMR multiplied by an appropriate time period, usually one day. (RMR is a rate of energy expenditure whereas REE is a total energy expenditure over a given time period, though REE and RMR are often both used to denote the energy expenditure per day due to resting metabolism). Conventional indirect calorimeters are too large and expensive to be used as part of a weight control program. Recently, James R. Mault M.D. et al. invented an improved indirect calorimeter, embodiments of which are well suited for applications in improved weight control and health management programs. The improved indirect calorimeter is more fully described in pending U.S. application Ser. No. 09/630,398.

Conventional diet calculators enable food records to be created on a hand-held-device. However, conventional devices are not used to store and transmit data to a remote computer system over a communications network. A conventional calorie management device does not comprise a glucose sensor, so that the blood glucose levels of the person can be monitored, and entering of foods consumed used to predict future blood glucose levels.

### SUMMARY OF THE INVENTION

As part of a physiological monitoring system, a person wears a wrist-mounted device, which has data storage, data receiving, and data transmission capabilities. The wrist-mounted device can include the functionality of a time-keeping device, and can resemble a wristwatch or wrist mounted computer.

The wrist-mounted device can receive and store data relating to the person wearing it, such as metabolic rate, body weight, body fat content, blood component analysis, respiration component analysis, heart monitoring, pulse rate, altitude, position (physical location), physical activity levels, and/or other physiological or medical data. In the preferred embodiment, the wrist-mounted device is used in conjunction with an indirect calorimeter, a device for measuring metabolic rate from respiratory analysis. An example of such an indirect calorimeter is the Gas Exchange Monitor (GEM), invented by James R. Mault et al., and described in U.S. application Ser. No. 09/630,398, and Int. App. WO001/08554A1 (published Feb. 08, 2001). An embodiment of the disclosed indirect calorimeter comprises a fluorescence-based oxygen sensor and a bi-directional ultrasonic flow meter to determine the oxygen consumption of a person, allowing metabolic rate of the person to be found. Indirect calorimeters using other gas flow sensors and gas component sensors can also be used. For example, IR absorption, IR emission, molecular fluorescence, surface adsorption effects, photoionization, electrochemical effects, paramagnetic sensors, electrical capacitance based sensors, colorimetric sensors, optical sensors, and other spectroscopy based sensors can be used for composition analysis of exhaled gases. Thermal (e.g. hot wire), and pressure-drop based flow sensors can be used for flow measurements. Ultrasonic density measurements to determine the density and hence composition of exhaled gases can also be used. Other metabolic rate meters, such as the Douglas bag and metabolic carts, can be used if available. However, the GEM is well suited for metabolic rate measurements.

A person may breath through an indirect calorimeter (preferably, a GEM), whilst wearing the wrist-mounted device according to the present invention. Respiration data from the indirect calorimeter is transmitted to the wrist-mounted device using, for example, a wireless link such as Bluetooth protocol, IEEE802.11, wireless Ethernet, ultrasound, IR, or other electrical/electromagnetic methods. In the preferred embodiment, a wireless communication protocol such as Bluetooth is used. The Bluetooth protocol can also be used for communication between the wrist-mounted device and other devices, such as a physical activity monitor, weighing scale, other physiological monitor, and the like. Cables, memory module transfer, and manual entry of data can also be used for data transfer. Calculation of metabolic rate from respiration data is preferably performed by the indirect calorimeter, but can also be calculated by the wrist-mounted device, or other devices such as a computer or interactive television, with which the wrist-mounted device communicates. The metabolic rate determined for the person is stored by the wrist-mounted device using electronic memory means.

The wrist-mounted device can also receive data from other sensors or devices, such as: body fat data (e.g. from electrical conductivity measurements); blood glucose concentration; physical activity (e.g. using one or more accelerometers, an accelerometer can be built into the wrist-mounted device); pulse rate, brain waves, heart function, respiration analysis, physical location using a global positioning system, etc. In the preferred embodiment, data are transmitted to the wrist-mounted device using a wireless link. However, the wrist-mounted device can also have data entry keys (e.g. a numeric keypad) for data entry. Sensors can be mounted into the wrist-mounted device, or elsewhere on the person's body. Transmission can use a wireless link (e.g. the Bluetooth protocol), IR, or other electrical/electromagnetic means. Sensors built into the wrist-mounted device will preferably have an electrical interface to data storage and/or data analysis circuitry. The wrist-mounted device can also receive data from devices not mounted on the person's body, e.g. other medical diagnostic or physiological monitoring equipment.

In a preferred embodiment, the wrist-mounted device also. includes a bar code reader for reading in information, such as printed bar codes on food packages. For example, nutrition information related to menu choices can be scanned at restaurants. Bar codes, nutrition information, or other codes can be printed on menus, receipts, or other papers, and scanned by the wrist-mounted device or accessory in communication with it. Data can be transmitted to the wrist mounted device by the food vendor, using IR or other wireless methods. A local wireless network can be used to transmit information inside a restaurant or other food retailer. Data can also be transmitted by vending machines, point-of-sale devices, or other devices.

Nutrition information on prepackaged foods, such as those bought from grocery stores or supplied in weight loss programs, can determined by reading a product identifier (e.g. UPC or universal product codes) and using the identifier to obtain dietary information from a database. The database can reside on the wrist-mounted device, or on a remote computer accessed by a communications network such as the Internet. The bar code reader circuitry can also be used for data entry, e.g. using time-modulated optical or IR signals. A pen-like device with optical character recognition can also be used to read identification or nutrition data from packaged foods and transmit it to the wrist-mounted device. A pen like device can also be used for bar-code scanning, with encoded data transmitted to the wrist-mounted device.

Nutrition information is often given in tabular form on a label attached to prepackaged foods. An imaging device can be used to capture an image of the label, with optical character recognition used to extract the nutritional information. Nutrition information can also be encoded by other methods. A bar code can be generated encoding nutritional information. A weight control business can label food products supplied to persons on a weight control program with a bar code which encodes nutrition information such as fat, fiber, carbohydrate, protein, vitamins, and other nutritional parameters' such as diet points (as used by Weight Watchers, UK), glycemic index, and the like. A number (or alphanumeric code) can be algorithmically generated from nutrition data, and entry of this number or code into a diet log program can be used for recording nutrition information, which can be algorithmically extracted from the entered data. Food products can also be labeled with transponders (such as inductively or capacitively coupled circuits), which provide nutrition or product identity data when close to the wrist mounted device.

The wrist-mounted device can also be used to provide feedback to the person. For example, it can help with meal choice decisions depending on person's progress with a weight loss program. It can use colored lights, audible tones, speech synthesis, or displayed characters. It can remind the person of scheduled activities such as a physician appointment, exercise, or meal times.

Data collected by wrist-mounted device can be transmitted to an electronic device with enhanced display capabilities, such as a computer, interactive television, personal digital assistant (PDA) etc. Data can also be transmitted using a communications network such as the Internet to remote computer(s) for storage in a database, or display to authorized persons via a web site. The wrist-mounted device can also have voice recognition and/or voice synthesis capabilities for communication purposes.

A person may sit in front of an interactive TV system, or other entertainment system. The wrist-mounted device communicates with the interactive TV using preferably an IR or wireless (e.g. Bluetooth) link. The interactive TV is linked via a communications network (e.g. the Internet) to a remote computer system. Data can be also be exchanged directly between the wrist-mounted device and the remote computer using a wireless Internet connection.

For example, a person on a weight control program wears the wrist-mounted device, which resembles a watch. The wrist-mounted device is used to display time, and on request, calorie balance. The calorie balance for a person is related to the calorie intake (e.g. from meals, snacks, etc.) compared with the calorie expenditure (calories expended by the person through resting metabolism, exercise, etc.). The person's metabolic rate is measured using a indirect calorimeter (e.g., the GEM) e.g. at weekly intervals, or as appropriate, and their activity level is estimated using one or more body-mounted accelerometers.

The person's calorie intake is recorded by the wrist-mounted device. Bar codes on pre-packaged foods are read by a bar-code reader in the wrist-mounted device, and converted to nutrition data using a database. If the person is eating prepackaged foods from a limited selection, e.g. meals supplied as part of a weight control program, or from a list of food items the person has bought before, the database relating bar-code data to nutrition information is most conveniently stored within memory means in the wrist-mounted device. This database is conveniently updated via the Internet. Other items having UPC (Universal Product Codes) are read by the bar code reader on wrist-mounted device, which then can communicates with a remote database using a wireless connection to a communications network such as the Internet, allowing nutrition data to be obtained from the UPC codes. Any internal database can be updated after information is received from a remote computer.

A database can also contain the UPC (Universal Product Codes) of approved foods, allowing the wrist-mounted device to help select such foods at a grocery store. Food products would be scanned, and the wrist-mounted device would indicate if the product was on an approved list, containing e.g. low fat foods. The wrist-mounted device can have a database relating the product identity or UPC code with nutrition information. The wrist-mounted device can also query a remote database over a communication network, such as a wireless Internet connection.

The following example illustrates how the invention can be used in a business model for a company selling weight control products and services. In this example, the person wearing the wrist-mounted device is referred to as the customer. The weight control company supplies the wrist-mounted device to the customer, for a fixed fee or rental payments. The customer has access to a indirect calorimeter for metabolic rate determination. The customer might be supplied with a indirect calorimeter, or a indirect calorimeter would be available elsewhere, e.g. at a fitness center, physicians office, store (e.g. drugstore), etc. A fitness center might provide access to a indirect calorimeter in order to attract customers. The indirect calorimeter is used to measure the metabolic rate of the customer, data that is stored in the wrist-mounted device. The preferred embodiment is for the indirect calorimeter to transmit the data to the wrist-mounted device using a wireless data transmission method, such as Bluetooth. Alternatively, the optical sensor in the bar code reader might be used to receive data transmitted using optical or IR radiation.

The weight loss company devises a weight control program for the customer, and supplies prepackaged meals. The meals can be charged to the customer separately, or might be part of an overall program billed to the customer. The customer uses the bar code reader to record the meals eaten using the codes on the packages. These codes are translated into nutrient information (e.g. calorie content, fat, carbohydrate, protein, fiber etc.) using a database, which is stored within the wrist-mounted device

The person weighs themselves at intervals, and the weight data is entered into the wrist-mounted device. The preferred method is for the weight to be transmitted to the wrist-mounted device using a wireless link. Suitably equipped scales can be supplied to the customer for a fee. Alternatively, conventional scales can be used and the data entered manually through numeric keys on the wrist-mounted device. The disadvantage with numeric entry is that errors can occur, or the customer can knowingly understate their weight.

Other devices will be offered to the customer by the weight loss company (for sale or rent), e.g. accelerometers for monitoring the person's activity, and/or other physiological sensors.

The wrist-mounted device also allows a person to exclude one or more a food item from their diet, for medical or cultural reasons, as described in the following example. For example, certain persons have serious peanut allergies. The barcode reader in the wrist-mounted device is used to read the UPC (Universal Product Code) bar code on a food item. If that item is known to contain peanuts, the device would warn the person, for example using an audible alarm and/or flashing red light. If the UPC code is not recognized, a yellow light might show. If the item is known to not contain peanuts, the green light would show. This use (to exclude food items from diet) is readily combined with a weight control use. For example, high fat items can be excluded from the person's diet.

An interactive TV or computer can be used to provide feedback to the person, as described in the following example. Data collected by the wrist-mounted device is transmitted to a device with display capabilities, such as an interactive television, computer, or other entertainment device. In the following example, an interactive television is used to provide feedback to the person. The interactive television receives data from the wrist-mounted device and provides a graphical display of dietary parameters. Caloric intake, activity, and weight might be shown, possibly as a function of time. Further data, or modifications to the data, can be carried out using the wrist-mounted device or a remote control for the interactive television.

The following example illustrates how physiological monitoring is implemented during a weight control program. A person on a weight control program can suffer a heart condition, which needs monitoring. The wrist-mounted device receives data from an additional heart monitor, such as an ECG sensor. Deviations from an accepted signal lead to a warning, and an alert to medical services via a wireless Internet connection.

The following example illustrates how blood sugar monitoring is implemented during a diet control program for a person with diabetes. The system described above is used, with the addition of a glucose sensor. The glucose sensor transmits data to the wrist-mounted device using a wireless link, preferably using the Bluetooth protocol. A glucose sensor could alternatively be built into the wrist-mounted device.

The wrist-mounted device is used to warn a person if a selected food item is likely to cause future deviations of blood sugar from a predicted range, using a current measurement of blood sugar level, the carbohydrate content of the food, and the blood sugar response of the person to certain foods. Food information is determined using a bar code reader on the wrist-mounted device reading UPC or other codes, of from nutrition information on the food package.

The system also recommends the optimum start time and duration for exercise, in order to maintain blood sugar levels within a certain range. It can be difficult for a person with diabetes to plan exercise, as blood sugar can drop dangerously low during the activity.

Hence, a weight control method for a person can comprise: carrying an electronic device having a data entry mechanism, a memory, a processor, and a display; measuring a metabolic rate using a metabolic rate meter such as an indirect calorimeter; transmitting the metabolic rate to the portable electronic device, so that the metabolic rate is recorded in the memory; recording food identifiers on the portable electronic device; recording physical activity estimates on the portable electronic device; and calculating an energy balance for the person, in terms of energy consumed as food, energy expended as physical activity, and energy expended as metabolism; and indicating the energy balance for the person on the portable electronic device, whereby the person is able to monitor the success of their dietary program.

Hence, a wrist-mounted device for assisting a person to maintain a calorie balance goal comprises: a memory; a processor; a display; a product identifier input mechanism, wherein product identifiers (such as barcodes, product names, identity codes, and the like) can be entered for foods consumed by the person. The wrist-mounted device further comprises an activity level input mechanism, which receives an activity signal correlated with the physical activity level of the person; a resting metabolic rate input mechanism for a resting metabolic rate as preferably determined using an indirect calorimeter; and a database correlating the product identifiers with calorie values for the foods consumed. The device further comprises a calorie management algorithm, adapted to calculate a calorie balance from the calorie values data correlated with foods consumed, the resting metabolic rate, and the activity level signal, and to present the calorie balance on the display. The activity level input mechanism can be a wireless transceiver adapted to receive an activity level signal from a body-mounted accelerometer, a bar code scanner, data entry keys, a stylus entry mechanism, a menu-based input system, or any other convenient data entry mechanism. The resting metabolic rate input mechanism can be communications link with an indirect calorimeter, for example a cable or wireless link. Alternatively, data can be manually entered into the device. The wrist-mounted device can further comprise a glucose sensor, adapted to provide a blood glucose measurement correlated with a blood glucose level of the person, so that the blood glucose measurement can be presented to the person on the display. The person can receives an alert (for example through an audible signal, flashing light, or vibration of the housing using conventional methods) when the blood glucose measurement of the person approaches the limits of a safe range.

Embodiment of a wrist-mounted device can assist a person maintain a blood glucose level within a safe range. The wrist-mounted device can comprise: a processor; a memory; a display; a glucose sensor, providing a glucose signal correlated with a blood glucose level of the person; a food identifier input, receiving food identifiers correlated with food items consumed by the person; and a software application program, executable by the processor, receiving the glucose signal, further receiving food identifiers, and having an algorithm adapted to calculate a future level of blood glucose for the person. The food identifier input can be a wireless transceiver, barcode scanner, one or more buttons, menu based input system, or other input mechanism as discussed elsewhere.

A system for calorie balance and blood glucose management, comprises: a body-mounted device, having a processor, memory, clock, a display, and an input mechanism for food identifiers correlated with consumables consumed; a metabolic rate meter in communication with the body-mounted device; a blood glucose meter in communication with the body-mounted device; and an algorithm, executable on the processor, adapted to receive a metabolic rate measurement from the metabolic rate meter, a blood glucose level from the blood glucose meter, and food identifiers from the input mechanism, and further adapted to calculate a current calorie balance for the person, and current blood glucose level of the person. The system can further comprise an activity monitor in communication with the body-mounted device, and an audio alert, for warning the person if their blood glucose level approaches the limit of a safe range.

The entire contents of tile following are incorporated herein by reference: U.S. provisional application Serial Nos. 60/207,05 1, filed May 25, 2000; 60/228,680, filed Aug. 29, 2000; 60/243,621, filed Oct. 26, 2000; 60/257,138, filed Dec. 20, 2000; and 60/269,063, filed Feb. 15, 2001; U.S. patent application Ser. Nos. 09/630,398, filed Aug. 2, 2000 and 09/745,373, filed Dec. 23, 2000; U.S. Pat. Nos. 6,135,107, 5,836,300, 5,179,958, 5,178,155, 5,038,792, and 4,917,108, and international application Nos. WO00/07498A1, published Feb. 17, 2000 and WO 01/08554A1, published Feb. 08, 2001.

### BRIEF DESCRIPTION OF THE DRAWINGS

FIG. 1 shows a schematic of a wrist-mounted device according to the present invention;

FIG. 2 shows a cross-sectional schematic of the device;

FIG. 3 shows a front view of the device;

FIG. 4 shows a calorie management system using the wrist-mounted device;

FIG. 5 shows a schematic of an activity monitor used in a calorie management system;

FIG. 6 is a flowchart illustrating a calorie management system;

FIG. 7 illustrates a method of blood sugar control;

FIG. 8 also illustrates a method of blood sugar control;

FIG. 9 is a schematic of a system including a portable computing device;

FIG. 10 illustrates a system including an interactive television system; and

FIG. 11 illustrates a system using a wireless telephone network.

### DETAILED DESCRIPTION OF THE PREFERRED EMBODIMENTS

FIG. 1 shows a schematic of a wrist-mounted device according to the present invention. The device comprises a processor 10, a display 12, a memory 14, a glucose sensor 16, a wireless transceiver 18, a barcode scanner 20, a data entry port 22, a data entry mechanism 24, a clock 26, an audio alert 28, and a light emitting diode 30.

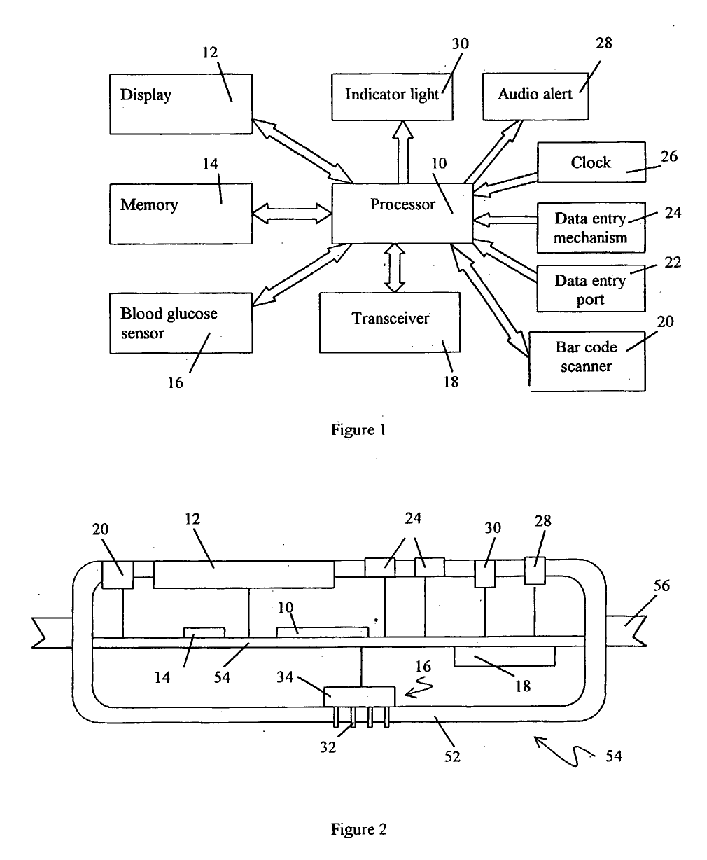

FIG. 2 shows a cross-sectional schematic of the wrist-mounted device 50, showing a housing 52, a circuit board 54, and strap 56. The processor 10, memory 14, and clock 26 are mounted on the circuit board 54. The display 12 is mounted on the front of the device, to be shown to the person wearing the device when it is strapped to a wrist. The barcode scanner 20, transceiver 18, LED 30, audio alert 28 and data entry buttons such as 24 are also shown on the top of the device. However, the location of the individual components is not essential. The glucose sensor 16 is mounted on the rear of the device, the bottom of the housing as shown on FIG. 2, so as to contact the skin of the person wearing the device. The glucose sensor 16 comprises an array of microcapillaries 32 adapted to withdraw interstitial fluid from the skin of the person into the analysis module 34. The interstitial fluid is drawn into the analysis module 34, which provides an electrical signal correlated with the glucose concentration in the interstitial fluid, and hence correlated with the glucose concentration in the blood. A suitable glucose sensor is disclosed in co-pending U.S. Provisional Application Serial No. 60/257,138 (filed Dec. 20, 2000) to James R. Mault. Other glucose sensors which can be advantageously used in the present invention are disclosed in U.S. Pat. Nos. 6,056,738, 6,152,889, 6,080,116, 5,820,570, 5,746,217, and 5,582,184, the entire contents of which are incorporated herein by reference.

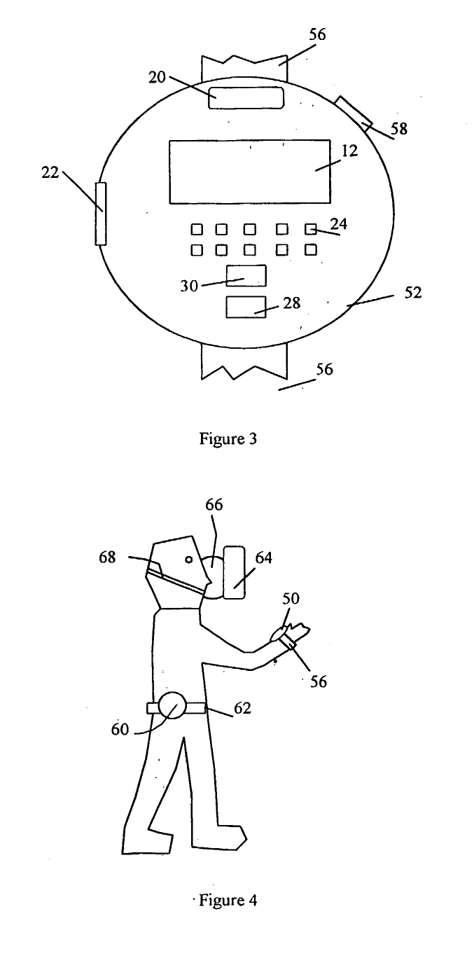

FIG. 3 illustrates a front view of the device, showing the strap 56 used to secure the device around the wrist of the wearer. The front view shows barcode scanner 20, display 12, LED 30, audio alert 28, and data entry buttons 24. Additional buttons such as 58 can be disposed around the side edges of the device, for example electrically connected to the processor to change the operational mode of the device (which might include time, diet records, entry of diet consumed, activities, metabolic rate, and other data, display of calorie balance, display of current and future predicted blood glucose levels, planning mode (such as entry of possible foods for receiving feedback on their suitability), and the like). A data entry port 22, which can be an electrical socket, IR or second wireless transceiver, is shown disposed at the side of the device. A low power transceiver can be used to receive data from physiological sensors, activity sensors, and other sensors disposed on, around, or implanted in the person's body.

In use, the glucose sensor provides measurements at intervals of the person's blood glucose level, which are analyzed by the processor and stored in the memory. The person uses the barcode scanner or other data entry mechanism such as buttons to enter product codes associated with food items consumed. In this specification the term “food items” refers to meals, vending machine products, beverages, nutraceuticals, and other products consumed by the person. The person can further enter a resting metabolic rate and an estimate of activity energy expenditure into the device, allowing a calorie management algorithm executed by the processor to calculate the current calorie balance of the person. If an entered product code is calculated to not meet a calorie balance goal, a warning indication or warning sound may be used to alert the person.

The device can also be used in blood glucose management systems. For example, the person may enter a product code or scan the barcode of a product that the person is planning to consume, and using the current blood glucose level and known glycemic response of the identified product, an alert may sound if the planned consumption will cause the person's blood glucose level to exceed a safe limit. The wrist-mounted device can be provided with an algorithm for predicting future levels of blood glucose, in terms of current blood glucose levels, insulin levels (which can also be measured using the device), known insulin response curves of the person to eating, the carbohydrate content of the food, and the glycemic index of the food.

FIG. 4 illustrates the components of an improved calorie management system incorporating a wrist-mounted device according to the above description. The wrist-mounted device 50 is shown secured to the wrist of a person using a strap 56. The person also carries a physical activity monitor 60 secured around the body of the person using strap 62. In other embodiments, the wrist-mounted device may be used as a body-mounted device, and combined with the activity monitor into a unitary device secured around the body of the person. In other embodiments, the wrist-mounted device can detect physiological indications of activity, and in these embodiments the wrist-mounted device can also be used as an activity monitor. For example, physical activity is correlated with body temperature and pulse rate. In this case, a pulse rate sensor and/or a temperature sensor may be included within the housing of the wrist-mounted device, so as to provide a signal correlated with the physical activity of the person.

The person is also shown breathing through a mask 66, in fluid communication with an indirect calorimeter 64, supported by strap 68. A mouthpiece can also be used to breath through the indirect calorimeter. The indirect calorimeter is preferably the gas exchange monitor (GEM) described by James R. Mault and others in U.S. patent application Ser. No. 09/630,398 (filed Aug. 2, 2000). Other metabolic rate meters can be used however, for example the indirect calorimeters described in U.S. Pat. Nos. 6,135,107, 5,836,300, 5,179,958, 5,178,155, 5,038,792, and 4,917,108, and PCT App. WO01/08554A1 (published Feb. 08, 2001). The metabolic rate of the person determined by the indirect calorimeter can be transmitted to the wrist-mounted device using a wireless link, such as a Blue Tooth protocol, local wireless network, IR link, or other wireless link. Alternatively, a cable can link the indirect calorimeter to the wrist-mounted device, so as to provide a metabolic rate reading to the wrist-mounted device. The data entry mechanism 24 can be used to manually enter metabolic data (or oxygen consumption data, VO₂), provided by the indirect calorimeter into the wrist-mounted device.

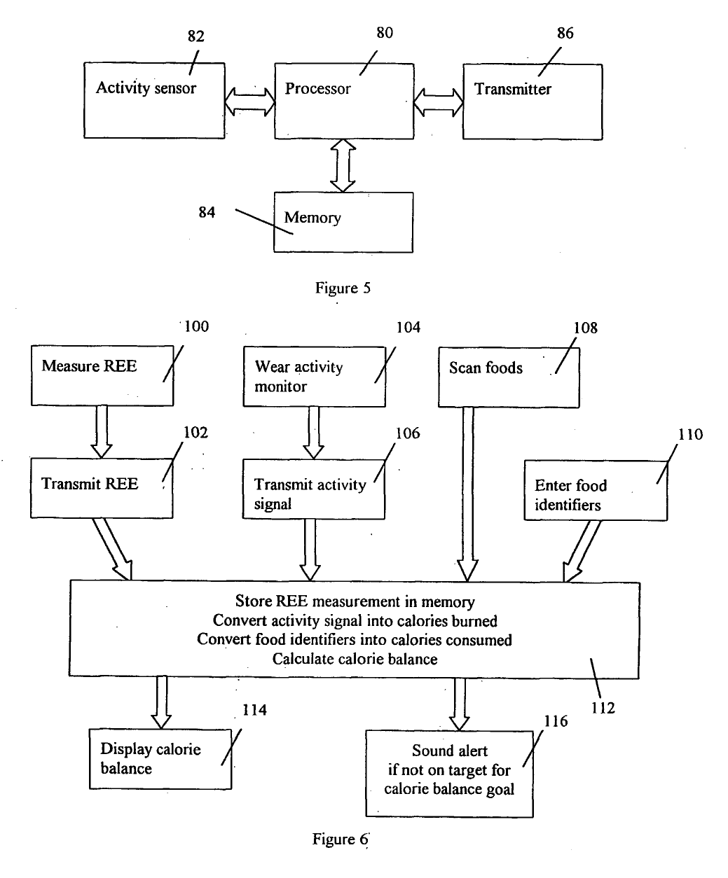

FIG. 5 shows a schematic of an activity monitor, such as 60 in FIG. 4, which can be used in a calorie balance system. The activity monitor comprises an activity sensor 82 providing an electrical signal correlated with physical activity level of the person, a processor 80, a memory 84, and a wireless transmitter (or transceiver) 86. The activity sensor 82 preferably is an acceleration sensor providing a signal correlated with the vertical component of acceleration of the person's torso. This can be correlated with the activity energy expenditure of the person using the indirect calorimeter. For example, the person may engage in activities of varying intensities while wearing an indirect calorimeter, allowing the signal from the activity monitor to be correlated with the metabolic rate determined by the indirect calorimeter. The activity sensor may also be a heart rate monitor, physical location device (such as a global positioning system, or sensor detecting repetitions of an exercise, or device receiving signals from an exercise machine.

Calorie Balance System

The wrist-mounted device can be used to monitor the calorie balance of a person. FIG. 6 illustrates a method for providing calorie balance feedback. Boxes 100-110 correspond to methods of generating and inputting calorie management data into the wrist-mounted device, Box 112 corresponds to the operation of an algorithm or software application program on the wrist-mounted device, and Boxes 114-116 correspond to provision of feedback to the person. Box 100 corresponds to the measurement of resting energy expenditure using a metabolic rate meter such as the indirect calorimeter. Box 102 corresponds to the transmission of the metabolic rate to the wrist-mounted device, for example using wireless or cable connections. Box 104 corresponds to providing the person with an activity monitor, the monitor preferably being supported by the person and providing a signal correlated with the physical activity levels of the person. Box 106 corresponds to the transmission of an activity signal to the wrist-mounted device. This may be done continuously, at intervals, by wireless, cable, or other electrical interface methods, or may be performed using the transfer of memory modules such as the Sony Memory Stick™, San Disk memory modules, or other solid state, magnetic, or optical memories. Box 108 corresponds to the scanning of food packages so as to determine a scan code, such as a barcode. Box 110 corresponds to the entering of product information into the wrist-mounted device by another mechanism, such as data entry using buttons. Box 112 corresponds to the function of a calorie management program running on the wrist-mounted device. This program converts the activity monitor signal to calories expended. The program also converts entered product identifications to calories consumed, using a database correlating food identity with calories consumed. The wrist-mounted device may also have a database correlating scan codes, such as Universal Product Codes (UPC) with product identification. After calculating calorie expenditure through activity and resting metabolic processes, and after calculating calorie intake through consumption, the program can then calculate the calorie balance for the person. Box 114 corresponds to a display of the balance to the person, which may be in numerical form on the display, or through colored lights, icons, animated graphics, or audio signals such as synthesized speech, buzzers, and the like. Box 116 corresponds to the indication of an alarm, and box 116 corresponds to the sounding of an alert if planned food consumption will cause calorie balance goals not to be met.

Before scanning or entering a food item identifier, the person can indicate whether the food has been consumed or is a planned meal. If tile food has been consumed, the calorie balance will be updated. If the person plans to consume a food item, the projected calorie balance can be displayed along with suggested alternatives if the item is not consistent with calorie management goals.

Blood Sugar Control

The wrist-mounted device is well suited for a combined function of blood glucose monitoring and calorie balance management.

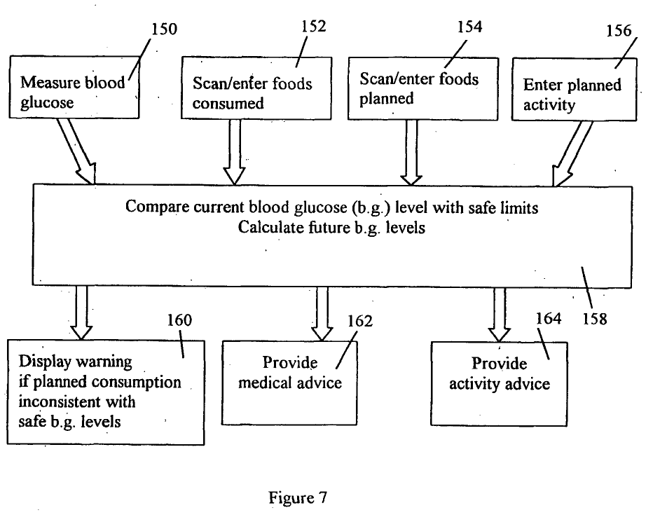

FIG. 7 illustrates schematically a method of simultaneously monitoring calorie balance and blood glucose levels. Box 150 corresponds to the measurement of blood glucose levels preferably using a glucose sensor within the wrist-mounted device. However, blood glucose levels can also be measured by other devices, such as other body-mounted sensors, which may transmit a signal correlated with the blood glucose level via a wireless link to the wrist-mounted device. Alternatively, conventional blood glucose meters can be used, and the data entered into the wrist-mounted device using the data entry mechanism.

Box 152 corresponds to the scanning or entering of foods consumed by the person. For example, the barcode scanner can be used to scan UPC codes on prepackaged foods. A portable computing device can also be used in assisting the person enter foods consumed. For example, a personal digital assistant may present a menu of food items to a person, which can then be selected and transmitted to the wrist-mounted device. A menu system as disclosed by Williams in U.S. Pat. Nos. 5,704,350 and 4,891,756, incorporated herein by reference, can be used for entering food identifiers corresponding to food or other consumables consumed. The bar code scanner may comprise an adjacent light emitter and detector, so that a modulated reflection of the emitted light is received by the detector as a bar code is moved in front of the bar code scanner. A hand-held scanner, for example in the form of a pen, or other scanner disposed conveniently on the person's body, can be used to scan items and transmit the data to the wrist-mounted device for example by a wireless transmission method.

Box 154 corresponds to the scanning or entering of planned food consumption, using methods described with respect to box 152. The operating mode of the wrist mounted device can be changed to a planning mode before entering planned meals.

Box 156 corresponds to the entering of activities or planned activities. An activity signal correlate with activity levels can be received from an activity sensor. The activity signal can be used to modify the projection of future blood glucose levels.

Box 158 corresponds to the calculation of a future blood glucose level based on the current measured level and the foods either consumed recently, or planned to be consumed. This may use a glycemic response algorithm, for example as disclosed in co-pending U.S. Provisional App. No. 60/269,063 (filed Feb. 15, 2001).

Box 160 corresponds to the provision to the person of a visual warning according to current and projected future blood glucose levels. For example, if current blood glucose levels are within a safe range, a first light (or icon on the display) may be green, but if future levels are projected to be outside of a safe range, a second light (or icon on the display) may be red. Animations, icons, graphics, bar charts, charts, graphs, and the like can be shown on the display 12 so as to provide feedback to the person.

Box 162 corresponds to the provision of medical advice to the person, for example advice on insulin injections, consumption of fibrous or other materials so as to slow the absorption of blood glucose, the desirability of exercise in light of current and projected blood glucose levels, and the like, based on the current and projected blood glucose levels. If the wrist-mounted device has a connection to a communications network, a medical professional may be contacted and provided with the current and projected data.

Box 164 corresponds to the provision of exercise advice. For example, if blood glucose levels are projected to be below safe limits, it may be inadvisable for the person to perform a planned exercise.

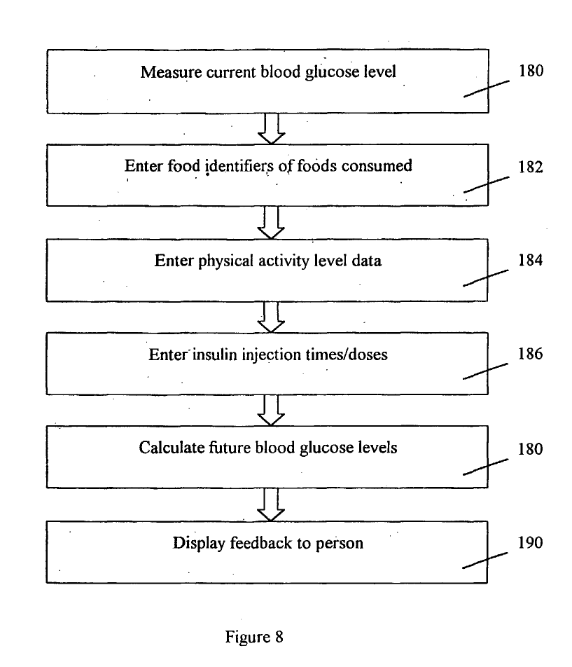

FIG. 8 illustrates a flowchart corresponding to the operation of a blood glucose management software application running on the wrist-mounted device (for example, as represented by Box 158 in FIG. 7). Box 180 corresponds to the measurement of current blood glucose levels. Box 182 corresponds to the entering of foods consumed. Box 184 corresponds to the entering of activity levels, and box 186 corresponds to the insulin injections (either future or present) of the person. (This step can be omitted if the person does not require insulin injections). Box 188 corresponds to the calculation of a future blood glucose level, and box 190 corresponds to the display of feedback to the person, for example in the form of a graph showing future behavior of blood glucose, with danger times in distinct color or otherwise highlighted.

System Including Portable Computing Device

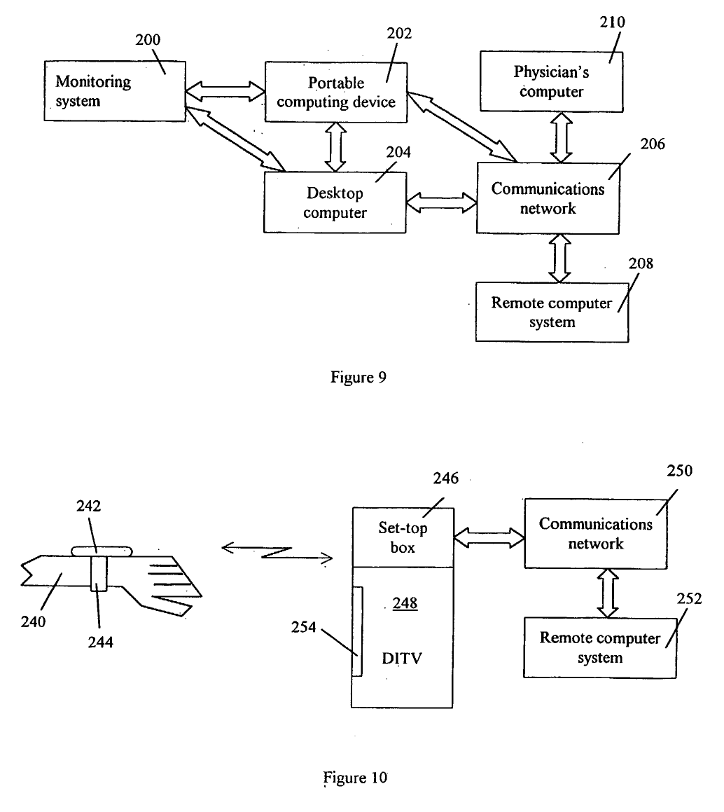

FIG. 9 shows a system embodiment including monitoring system 200, a portable computing device 202, a desktop computer system 204, a communications network 206, a remote computer system 208, and a physician's computer 210. The monitoring system may comprise the wrist-mounted device disclosed in previous examples. The wrist-mounted device has a display, a processor, a clock, and a memory. The device can further comprise a glucose sensor, a product code scanner such as a barcode scanner, an activity sensor, a wireless transceiver, an IR transceiver, and other components. The monitoring system may further comprise a separate glucose sensor, which communicates either with the wrist-mounted device or with the portable computer, preferably using a wireless link. The monitoring system may comprise a separate handheld product code scanner, such as a barcode scanner, adapted to transmit scanned codes either to the wrist-mounted device or to the portable computer. The monitoring system can comprise a separate glucose sensor, or a conventional test kit, whereby measured blood glucose can be entered into the wrist-mounted device or PDA.

The portable computing device 202 is preferably a personal digital assistant (PDA) such as manufactured by Palm Computing, Handspring, or PocketPC devices running the Microsoft operating system. The portable computing device may be any portable electronic device having a display and computing capabilities.

Data can be transferred from the wrist-mounted device or from the portable computing device to a desktop computer (personal computer) system 204. Data can further be communicated between the monitoring system, portable computing device, and desktop computer system, with a remote computer system over the communications network. Data can also be transmitted to the physician's computer 210. The desktop computer system may be in the person's home, or at a diet clinic, gym, or other health, weight, fitness, diet, vending, sport, or physiology related location.

Data collected by the monitoring system can be transmitted to another computer system for analysis and provision of feedback. Communication links are shown by double arrows. Communication links may comprise wireless, optical, cable, or data transfer links. The monitoring system can also communicate with the communications network, for example through a wireless Internet connection.

Interactive Television System

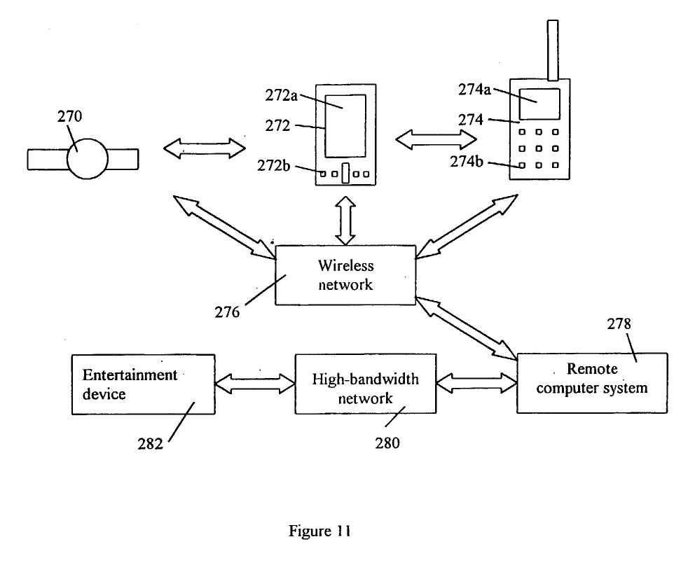

FIG. 10 shows a health monitoring system including an interactive television and a wrist-mounted device. The person 240 wears a wrist-mounted device 242 secured around a wrist of the person 240 using strap 244. The device comprises a transceiver for communicating with set top box 246. Set top box 246 has a communications link to a communications network 250 allowing data to be exchanged with a remote computer system 252. The remote computer system can be used to provide content for display on the interactive television 248. The remote computer system may also have a computer expert system for provision of health advice to the person. In use, the person uses the wrist-mounted device to collect health, diet, activity, weight, physiological and/or other information and transmits it to the set top box using a wireless communications link, such as the Bluetooth protocol, or an IR communications method. The data received by the set-top box is transmitted to the remote computer for analysis and provision of feedback. For example, if the person's blood glucose level has exceeded a safe range, the feedback may comprise advice on improved control of blood glucose levels. The display 254 of the interactive television 248 can be used to show, for example, diet or health related videos to the person, charts, graphs, and the like.

The wrist-mounted device may comprise a temperature sensor, and can be used to remotely monitor the temperature of a child. Temperature data can be transmitted to a remote computer system. A temperature sensor can be disposed elsewhere on the child's body, and can transmit data to the wrist-mounted device, for re-transmission to a remote device.

The wrist-mounted device can also be used as a remote control for operation of the interactive television system. Alternatively, data can be transferred from the wrist-mounted device to a suitably adapted remote control, which can then be used for control and interaction with the television system, and for the transmission of data to the remote computer system. For example, data collected by the wrist-mounted device may be transferred to a remote control by an electrical interface, and the remote control used to transmit the data to the set-top box using IR transmission.

FIG. 11 illustrates schematically communication between the wrist-mounted device 270 and portable computing device 272, and between these devices and a remote computer system 278. Data can be transmitted to a wireless phone 274 and transmitted over a wireless telephone network to the remote computer system 278. The portable computing device, and wrist-mounted device, may comprise the functionality of a wireless phone, in which case data can be transmitted directly over the wireless phone network to the remote computer system 278. Feedback can be returned over the wireless telephone network 276. However, a faster communications network 280 can be used to provide information rich content to the person over audiovisual display 282. This display may be part of an interactive television system as discussed above.

The described embodiments of the wrist-mounted device can be adapted to be supported by other parts of the person's body, for example by the waist or torso using a strap.

The invention is not to be limited by the above examples. Having described our invention, we claim:

1. A wrist-mounted device for assisting a person to maintain a calorie balance goal, the device comprising:
a memory;
a processor;
a display;
a product identifier input mechanism, wherein product identifiers correlated with foods consumed by the person can be input into the device;
an activity level input mechanism, adapted to receive an activity signal correlated with the physical activity level of the person;
a resting metabolic. rate input mechanism, wherein the person can enter a resting metabolic rate;
a database, residing in the memory, correlating the product identifiers with calorie values; and
a calorie management algorithm, adapted to calculate a calorie balance from the calorie values correlated with foods consumed, the resting metabolic rate, and the activity signal, and to present the calorie balance on the display.

2. The wrist-mounted device of claim 1, wherein the activity level input mechanism is a wireless transceiver adapted to receive an activity signal from a body-mounted activity monitor.

3. The wrist-mounted device of claim 1, wherein the product identifier input mechanism comprises a bar code scanner.

4. The wrist-mounted device of claim 1, wherein the resting metabolic rate input mechanism comprises a communications link with an indirect calorimeter.

5. The wrist-mounted device of claim 1, further comprising a glucose sensor adapted to provide a glucose signal correlated with a blood glucose level of the person, whereby the blood glucose level can be presented to the person on the display.

6. The wrist-mounted device of claim 1, further comprising an alert, whereby the person receives an alert when the blood glucose level of the person approaches a limit of a medically acceptable range.

7. A wrist-mounted device for assisting a person maintain a blood glucose level within a safe range, the device comprising:
a processor;
a memory;
a display;
a glucose sensor, providing a glucose signal correlated with a blood glucose level of the person;
a food identifier input, receiving food identifiers correlated with food items consumed by the person;
a software application program, executable by the processor, receiving the glucose signal, further receiving food identifiers, and adapted to calculate a future level of the blood glucose level for the person.

8. The wrist-mounted device of claim 7, wherein the food identifier input is a barcode scanner.

9. The wrist-mounted device of claim 7, wherein the food identifier input is a wireless transceiver.

10. The wrist-mounted device of claim 7, wherein the food identifier input comprises one or more buttons.

11. A system for calorie balance and blood glucose management, the system comprising:
a body-mounted device, having a processor, memory, clock, a display, and an input mechanism for food identifiers correlated with consumables consumed;
a metabolic rate meter in communication with the body-mounted device;
a glucose meter in communication with the body-mounted device, providing a glucose signal correlated with the blood glucose level of the person; and
an algorithm, executable on the processor, adapted to receive a metabolic rate measurement from the metabolic rate meter, a glucose signal from the glucose meter, and food identifiers from the input mechanism, and further adapted to display a current calorie balance for the person, and the blood glucose level of the person.

12. The system of claim 11, further comprising an activity monitor in communication with the body-mounted device.

13. The system of claim 11, further comprising an audio alert, wherein the person receives a warning if the blood glucose level approaches the limit of a safe range.
